(19) United States
(12) Patent Application Publication
(10) Pub. No.: US 2024/0201164 A1
(43) Pub. Date: Jun. 20, 2024

(54) EVALUATION OF PATIENTS WITH CYSTIC FIBROSIS USING SWEAT

(71) Applicant: THE RESEARCH INSTITUTE AT NATIONWIDE CHILDREN'S HOSPITAL, Columbus, OH (US)

(72) Inventors: Don Hayes, Columbus, OH (US); Fred Woodley, Columbus, OH (US); Ben Kopp, Columbus, OH (US)

(21) Appl. No.: 18/288,008

(22) PCT Filed: Apr. 21, 2022
(86) PCT No.: PCT/US2022/025691

§ 371 Date: Oct. 23, 2023

Related U.S. Application Data
(60) Provisional application No. 63/178,422, filed on Apr. 22, 2021.
(60) Provisional application No. 63/178,791, filed on Apr. 23, 2021.

(51) Int. Cl. G01N 33/487; G01N 30/02; G01N 30/88

(52) U.S. Cl. CPC G01N 33/487; G01N 30/88; G01N 2030/027; G01N 2030/884; G01N 2800/12; G01N 2800/382; G01N 2800/50

(57) ABSTRACT

A method of determining if a patient having cystic fibrosis has an increased risk of having or developing pulmonary exacerbations is described. The method includes determining the level of one or more metabolites associated with pulmonary exacerbations in a sweat sample from the patient, and characterizing the patient as having an increased risk of having or developing pulmonary exacerbations if one or more metabolites associated with pulmonary exacerbation are significantly different from a control value. A method of evaluating the response of a patient having pulmonary exacerbations to treatment is also described.

### CROSS-REFERENCE TO RELATED APPLICATIONS

This application claims priority to U.S. Provisional Application Ser. No. 63/178,422, filed on Apr. 22, 2021, and U.S. Provisional Application Ser. No. 63/178,791, filed on Apr. 23, 2021, both of which are hereby incorporated by reference in their entirety.

### BACKGROUND

Cystic fibrosis (CF) is a multisystem, genetic disorder affecting the respiratory, digestive, endocrine, immune, and reproductive systems. With abnormal chloride and sodium transport across secretory epithelia, people with CF (PWCF) develop thick, viscous secretions that ultimately alter organ function. Ratjen F, Doring G., Lancet. 361:681-9 (2003). Although CF is a systemic disease affecting numerous organ systems, progressive lung disease continues to be the major cause of morbidity and mortality for most PWCF. With altered rheology of airway secretions, clearance of secretions becomes more difficult, which leads to chronic obstruction of the smaller airways and the development of chronic infection by pathogenic bacteria and bronchiectasis with tissue destruction.

The clinical course for PWCF is complicated by frequent periods of acute deterioration in their pulmonary health which is often referred to as a “pulmonary exacerbation” (PEx). Although a consensus is lacking on the definition of PEx in CF, there are commonly accepted clinical parameters that are indicative of an acute PEx episode, which are treated with enteral or parenteral antimicrobial therapy targeting the bacterial pathogens present on respiratory cultures. Sanders et al., Pediatr Pulmonol., 46:393-400 (2011). Upon presentation with PEx, clinical symptoms in PWCF may include new onset or worsening cough, sputum production, dyspnea, fatigue, reduction in appetite, alterations in the color and/or consistency of sputum, or poor exercise tolerance. A minority of PWCF may present with fever. Upon clinical evaluation, clinical signs consistent with a PEx include reduced pulmonary function, specifically the forced expiratory volume in 1 second (FEV1), weight loss, or new onset or worsening crackles on physical exam, whereas a reduction is oxyhemoglobin saturation (SaO₂) or changes on chest radiographic may help support the diagnosis of a PEx but are not required. In epidemiologic studies, decreased FEV1% predicted is the most consistent predictor of PEx occurrence. Andrinopoulou et al., BMC Pulm Med., 20:142 (2020).

Metabolomics is an evolving field that has been used in CF for a variety of purposes, from understanding therapeutic responses to monitoring longitudinal changes in bacterial metabolism. Muhlebach M S, Sha W., 2:9 (2015); Quinn et al., PeerJ., 4:e2174 (2016). In particular, several prior CF studies have quantitated metabolites in sputum (Hahn et al., 10:174 (2020)), exhaled breath condensate (Zang X, Monge M E, Gaul D A, McCarty N A, Stecenko A, Fernandez F M. Early Detection of Cystic Fibrosis Acute Pulmonary Exacerbations by Exhaled Breath Condensate Metabolomics. J Proteome Res. 2020; 19:144-52), and plasma to help better characterize physiologic changes during PEx. Each of these sample types has collection limitations such as ease of access in all age groups (e.g. exhaled breath condensates, sputum, and blood) or invasiveness (blood). Further, with the increasing use of highly effective cystic fibrosis transmembrane conductance regulator (CFTR) modulators (Middleton P G, Mall M A, Drevinek P, Lands L C, McKone E F, Polineni D, et al., The New England journal of medicine, 381:1809-19 (2019)) that decrease sputum production and slow rate of decline in FEV1 (Sawicki et al., Am J Respir Crit Care Med., 192:836-42 (2015)), new non-invasive methods to monitor PEx treatment and resolution are needed. Sweat is a non-invasive biofluid that can be collected in any person with CF regardless of age or disease condition. Sweat is also routinely used in the diagnosis of CF. Although sweat has been previously used in a metabolomics study of newborn-screen positive infants with CF (Macedo et al., ACS Cent Sci., 3:904-13 (2017)), sweat metabolomes have not been examined during PEx.

### SUMMARY

People with cystic fibrosis (PWCF) suffer from acute and often unpredictable declines in clinical status known as pulmonary exacerbations (PEx). PEx symptoms vary between persons and within an individual over time; without universal diagnostic criteria, prediction and/or timely diagnosis is difficult. Lack of sensitivity in diagnosing and predicting PEx is accentuated by the fact that repeated PEx are disproportionally responsible for morbidity and mortality in CF.

The inventors sought to characterize the sweat metabolome in PWCF associated with an acute PEx prior to and then after completion of intravenous antibiotic treatment 1) to help define biological processes occurring during the PEx and 2) develop non-invasive biomarkers predictive of clinical response. To provide this characterization, PWCF were recruited and sweat samples collected using the Marcoduct® Sweat Collection System at the time of hospitalization and prior to discharge. Samples were analyzed for metabolite changes using ultra-high-performance liquid chromatography/tandem accurate mass spectrometry (UHPLC/MS-MS).

Twenty-six of 29 PWCF completed the entire study. A total of 326 compounds of known identity were detected in sweat samples. Of detected metabolites, 147 were significantly different between pre- and post-PEx samples (average treatment 14 days). Overall, sweat metabolomes changed from time of enrollment and post-treatment. Moreover, metabolomic changes were similar in PWCF who failed to return to baseline pulmonary function and those who did not. The inventors also discovered targeted metabolite profiles associated with predictive of PEx status, but not failure to return to baseline lung function. The findings demonstrate the feasibility of non-invasive sweat metabolome profiling in PWCF and defined metabolite profiles and biologic pathways that can be used in further research into preventative and therapeutic PEx strategies.

### BRIEF DESCRIPTION OF THE FIGURES

The accompanying drawings, which are incorporated in and constitute a part of this specification, illustrate some embodiments of the inventions, and together with the description, serve to explain principles of the inventions.

FIGS. 1A-1C provide graphs showing sweat metabolomics differentiates PEx states. A) 3D PLS-DA plots of overall metabolomics profiles between PWCF pre-PEx (red dots) and the same individuals post-PEx treatment (green dots). Model accuracy 0.84 with R2 0.85, cross validation by 10-fold CV. B) Loadings plot of the top 15 metabolites for the top component in the PLS-DA analysis in FIG. 1A comparing pre- and post-PEx profiles. A spectrum of metabolite expression intensity is displayed, with metabolites that are most over-expressed are shown in red, and under-expressed shown in green. Metabolites are plotted by their increasing importance to separation of profiles from top to bottom. C) Heat map of differentially expressed metabolites for PWCF pre-(red) and post-PEx (green). Every vertical column is an individual with grouping by hierarchical clustering using the average clustering algorithm with a ward's distance measure. The top 25 metabolites are displayed in the horizontal columns with differential expression shown in the color-coded expression intensity legend.

FIGS. 2A & 2B provide graphs showing alterations in sweat metabolite classes during PEX. A) Random forest biochemical importance plot of metabolites important for discriminating between post- vs pre-PEx. The top 30 biochemicals are presented in order of increasing importance to group separation with groupings of metabolites by color coding. A Random Forest Confusion Matrix of predicted classification for pre- vs post-PEx is shown with a predictive accuracy of 79%. B) Pathway enrichment analysis and pathway topology analysis (degree centrality and betweenness centrality) for sweat metabolomes pre- vs post-PEx. Metabolites are clustered in nodes with pathway impact on the X axis calculated by pathway topology analysis and plotted according to −log (p) values on the Y axis. Pathways are shown as nodes with varying significance based upon color and size. Post-PEx profiles were associated with changes in pyrimidine, phenylalanine, glycerolipids, glycine, taurine, arginine, and alanine metabolism.

FIG. 3 provides a graph showing Laol and IL-37 are upstream regulators of metabolic pathways during PEx treatment. Pathway analysis via IPA was performed using significantly altered metabolites between pre- and post-PEx groups. The top two regulators (Laol and IL-37) and their downstream effect networks are shown. A color-coded prediction legend displays predicted activation/inhibition of pathways as well as intensity of metabolite measurements.

FIGS. 4A-4C provide graphs showing sweat metabolomes predict enriched pathways and biomarkers of PEx. A) Metabolite Set Enrichment Analysis (MESA) of the top 50 pathways generated utilizing 99 metabolic pathway-associated metabolite reference sets for pre- vs post-PEx states. Quantitative enrichment analysis performed using GlobalTest. Pathways are plotted by fold enrichment in descending order. B) A multivariate exploratory receiver operative curve (ROC) for pre- vs post-PEx metabolite profiles was generated with linear support vector machine (SVM) analysis utilizing Monte-Carlo cross validation (MCCV) with balanced sub-sampling. Estimated false positive (x axis) and true positive (y axis) rates are shown for between 5-100 variables. The number of variables used are differentiated by color coding in the legend with area under the curve (AUC) and confidence intervals (CI) shown for each set. C) ROC performance was measured via a confusion matrix with the corresponding selected frequency of the top 15 metabolites shown. The confusion matrix demonstrated that the ROC analysis accurately distinguished pre- vs post-PEx states.

FIGS. 5A-5D provide graphs showing sweat metabolomes predictive of failure to return to baseline lung function following PEx. A) Changes in lung function (percent predicted forced expiratory volume in 1 second [FEV1]) for PWCF at their baseline and pre- and post-PEx treatment. FEVI changes are grouped by individuals who returned to within 5% of their 1-year average baseline FEVI (return to baseline) and those who failed to return to baseline. Each dot represents an individual with lines connecting all time points for each individual. Statistical significance via paired t-test. “**”=p value<0.01, “***”=p value<0.001. B) Heat map of differentially expressed metabolites for PWCF who return (green) and failed to return (red) to baseline FEV1 following PEx treatment. Every vertical column is an individual with grouping by hierarchical clustering using the average clustering algorithm with a ward's distance measure. The top 25 metabolites are displayed in the horizontal columns with differential expression shown in the color-coded expression intensity legend. C) 3D PLS-DA plots of overall metabolomics profiles between PWCF who return (green dots) and failed to return (red dots) to baseline FEV1. Model accuracy 0.58 with R2 0.99, cross validation by 10-fold CV. D) Loadings plot of the top 15 metabolites for the top component in the PLS-DA analysis in FIG. 5C comparing FEVI profiles A spectrum of metabolite expression intensity is displayed, with metabolites that are most over-expressed are shown in red, and under-expressed shown in green. Metabolites are plotted by their increasing importance to separation of profiles from top to bottom.

FIGS. 6A-6C provide graphs showing sweat metabolomes fail to accurately predict biomarkers of return to baseline FEV1. A) MESA analysis of the top 50 pathways generated utilizing 99 metabolic pathway-associated metabolite reference sets for return or fail to return to baseline FEV1 post-PEx. Quantitative enrichment analysis performed using GlobalTest. Pathways are plotted by fold enrichment in descending order. B) A multivariate exploratory ROC for post-PEx FEV1 metabolite profiles was generated with linear SVM analysis utilizing MCCV with balanced sub-sampling. Estimated false positive (x axis) and true positive (y axis) rates are shown for between 5-100 variables. The number of variables used are differentiated by color coding in the legend with AUC and CI shown for each set. C) ROC performance was measured via a confusion matrix with the corresponding selected frequency of the top 15 metabolites shown. The confusion matrix showed that the ROC analysis did not accurately distinguish FEV1 post-PEx states.

### DETAILED DESCRIPTION

The present inventions will now be described by reference to some more detailed embodiments, with occasional reference to the accompanying drawings. These inventions may, however, be embodied in different forms and should not be construed as limited to the embodiments set forth herein. Rather, these embodiments are provided so that this disclosure will be thorough and complete, and will fully convey the scope of the inventions to those skilled in the art.

Unless otherwise defined, all technical and scientific terms used herein have the same meaning as commonly understood by one of ordinary skill in the art to which these inventions belong. The terminology used in the description of the inventions herein is for describing particular embodiments only and is not intended to be limiting of the inventions. As used in the description of the inventions and the appended claims, the singular forms “a,” “an,” and “the” are intended to include the plural forms as well, unless the context clearly indicates otherwise.

The numerical ranges and parameters setting forth the broad scope of the inventions are in some cases approximations. Nonetheless, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, the term “diagnosis” can encompass determining the nature of disease in a subject, as well as determining the severity and probable outcome of disease or episode of disease and/or prospect of recovery (prognosis). “Diagnosis” can also encompass diagnosis in the context of rational therapy, in which the diagnosis guides therapy, including initial selection of therapy, modification of therapy (e.g., adjustment of dose and/or dosage regimen), and the like.

The terms “subject,” and “patient” are used interchangeably herein, and generally refer to a mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. Diagnosis of humans is of particular interest.

### Methods of Diagnosing Pulmonary Exacerbations

In one aspect, the present invention a method of determining if a patient having cystic fibrosis has an increased risk of having or developing pulmonary exacerbations. The method includes the steps of determining the level of one or more metabolites associated with pulmonary exacerbations in a sweat sample from the patient, and characterizing the patient as having an increased risk of having or developing pulmonary exacerbations if the level of one or more metabolites associated with pulmonary exacerbation are significantly different from a control value.

Cystic fibrosis (CF) is a multisystem, genetic disorder affecting the respiratory, digestive, endocrine, immune, and reproductive systems. Cystic fibrosis is inherited in an autosomal recessive manner, and is caused by the presence of mutations in both copies of the gene for the cystic fibrosis transmembrane conductance regulator (CFTR) protein. O'Sullivan B P, Freedman S D, Lancet. 373 (9678): 1891-904 (2009). Symptoms of cystic fibrosis include difficulty breathing and frequent lung infections, sinus infections, poor growth, fatty stool., clubbing of the fingers and toes, and infertility in most males.

Individuals suspected of having CF can be diagnosed using a sweat test and/or genetic testing. In addition, newborns can be screened for cystic fibrosis using a blood test for high levels of immunoreactive trypsinogen. For the sweat test, an electric current is used to drive pilocarpine into the skin, stimulating sweating. The sweat is collected and analyzed for salt levels. Having unusually high levels of chloride in the sweat suggests CFTR is dysfunctional. Genetic testing screens for CFTR mutations typically associated with CF.

The method is directed to determining if a patient having cystic fibrosis has an increased risk of having or developing pulmonary exacerbations. Patients having cystic fibrosis typically have frequent periods of acute deterioration in their pulmonary health which can be referred to as a “pulmonary exacerbation” (PEx). Commonly accepted clinical parameters that are indicative of an acute PEx episode have been described. See C. H. Goss, Semin Respir Crit Care Med., 40(6):792-803 (2009). Definitions generally combine patient reported symptomatology, laboratory data (particularly spirometry) and a clinician-based evaluation of the patient with the addition of a physician decision to treat the event.

The method includes determining the level of one or more metabolites associated with pulmonary exacerbations in a sweat sample from the patient. In some embodiments, the method further comprises the step of obtaining a sweat sample from the patient.

Sweat is the fluid secreted by the sweat glands in the skin of mammals. Whole body sweat is a complex mixture of cumulative secretions from millions of eccrine, apocrine, apoeccrine and sebaceous glands as well as bacteria, yeast, fungi, other microbiota and cellular debris present on the skin. Sweat is primarily water, typically has a moderately acid to neutral pH, and includes trace amounts of minerals, lactic acid, urea, and other metabolites (e.g., alcohols, peptides & proteins) such as the metabolites detected in the methods described herein. The main minerals are sodium, potassium, calcium, and magnesium.

Sweat samples can be obtained by any known means. Hussain et al., Clin Biochem Rev, 38(1):13-34 (2017). For example, sweat can be sampled and sensed non-invasively and continuously using portable devices such as electronic tattoos, bands, or patches. Heikenfeld, J., Electroanalysis. 28 (6): 1242-1249 (2016). The sweat sample can be a normal aqueous sample, or it can be dried. In some embodiments, the sweat sample is obtained using a skin patch. Sweat collection systems designed specifically for diagnosis of cystic fibrosis can also be used (e.g., the Macroduct® sweat collection system). A sweat sample may be fresh or stored. Samples can be stored for varying amounts of time, such as being stored for an hour, a day, a week, a month, or more than a month.

The method also includes determining the level of one or more metabolites associated with pulmonary exacerbations in the sweat sample. A variety of methods can be used to determine the metabolites present in a sweat sample, including gas chromatography, mass spectrometry, NMR spectroscopy, and high performance liquid chromatography. In some embodiments, multiple methods of analysis are used. For example, in one embodiment, the purified extract is divided into five fractions: two for analysis by two separate reverse phase Ultrahigh Performance Liquid Chromatography-Tandem Mass Spectroscopy (RP/UPLC-MS/MS) methods with positive ion mode electrospray ionization (ESI), one for analysis by RP/UPLC-MS/MS with negative ion mode ESI, one for analysis by HILIC/UPLC-MS/MS with negative ion mode ESI, and one sample was reserved for backup. In some embodiments, the level of the one or more metabolites is determined using high performance liquid chromatography. Methods of sample preparation are known to those skilled in the art. For example, Proteins may precipitated with methanol under vigorous shaking for several minutes followed by centrifugation to remove protein, to dissociate small molecules bound to protein or trapped in the precipitated protein matrix, and to recover chemically diverse metabolites.

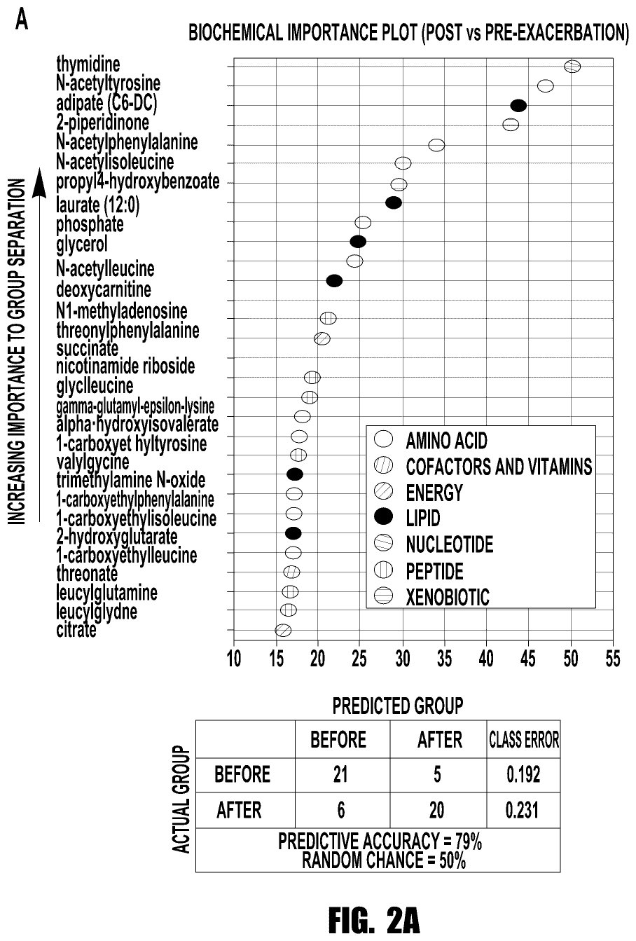

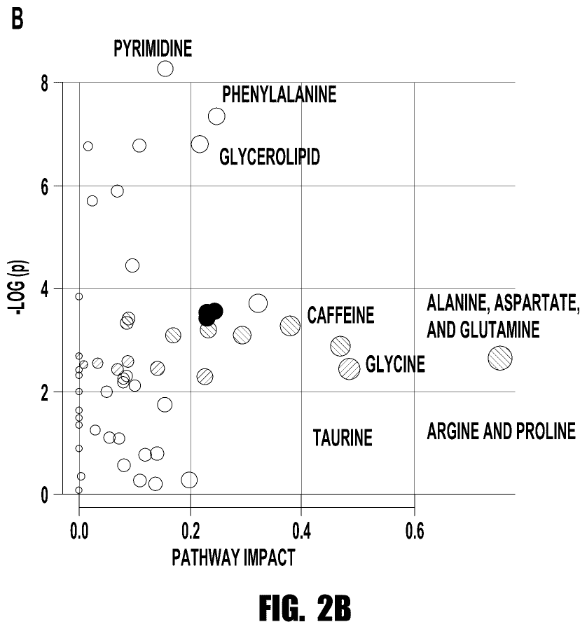

The inventors have identified the metabolites associated with pulmonary exacerbations. These metabolites are shown in FIG. 2. A variety of different types of metabolites were identified as being useful for diagnosing pulmonary exacerbations, including amino acids, cofactors and vitamins, energy molecules, lipids, nucleotides, peptides, and xenobiotics. The 30 identified metabolites are shown FIG. 2A, with the increasing importance of the metabolites shown on the Y axis of the graph. The metabolites used in the method can include any of the metabolites shown in FIG. 2A, however, it is preferable to use metabolites identified as being of higher importance. For example, in some embodiments, the metabolites associated with pulmonary exacerbations are the top metabolite (i.e., thymidine), the top 2 metabolites (i.e., thymidine and N-acetyltyrosine), the top 3 metabolites (i.e., thymidine, N-acetyltyrosine, and adipate (C6-DC), or the top 4 metabolites (i.e., thymidine, N-acetyl tyrosine, 2-piperidinone, and adipate C6-DC). In other embodiments, the top 5, top 6, top 7, top 8, top 9, top 10, top 11, top 12, top 13, top 14, top 15, top 16, top 17, top 18, top 19, top 20, top 21, top 22, top 23, top 24, top 25, top 26, top 27, top 28, top 29, or all 30 metabolites can be used. In addition, in some embodiments, metabolites from different regions of FIG. 2A can be used. For example, one could use a combination of thymidine, N-acetylisoleucine, and phosphate as metabolites for characterizing the risk of having or developing pulmonary exacerbations.

The risk aspect of characterizing a patient as having an increased risk of having or developing pulmonary exacerbations refers to a patient having a higher risk of having or developing pulmonary exacerbations in comparison with an average subject, and average patient, or an average patient having cystic fibrosis. The risk of having pulmonary exacerbations refers to a patient currently having pulmonary exacerbations that may not yet have been identified, while the risk of developing pulmonary exacerbations refers to a patient who does not currently have pulmonary exacerbations, but has an increased risk of developing pulmonary exacerbations in the near future (e.g., within a day or a week).

A patient is characterized as having an increased risk of having or developing pulmonary exacerbations if the level of one or more metabolites associated with pulmonary exacerbation are significantly different from a control value. The control value represents the value of a patient not having pulmonary exacerbations, such as a healthy subject, or a normal patient having cystic fibrosis but not pulmonary exacerbations. Control values can be obtained from the scientific literature, or can be obtained by carrying out a metabolite analysis using the methods described herein.

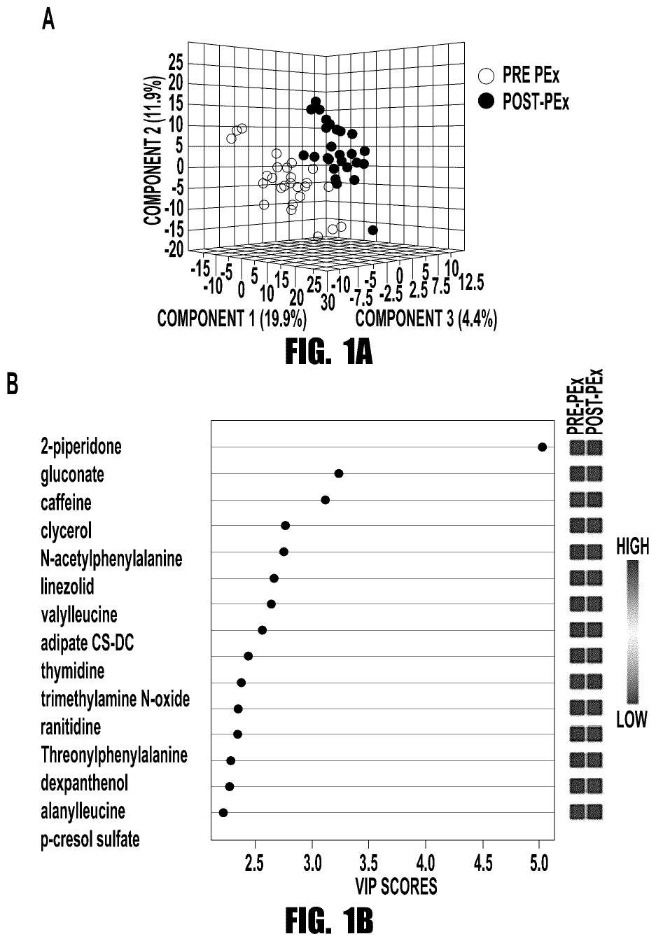

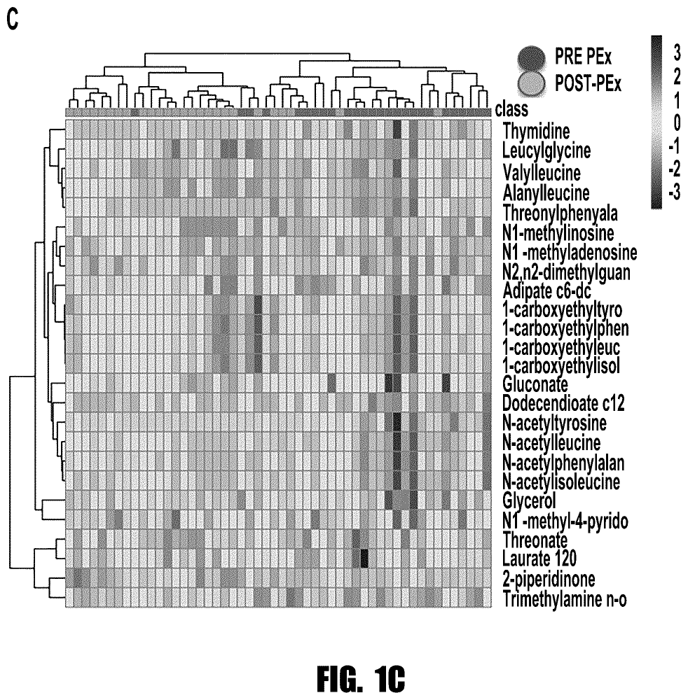

Depending on the metabolite, a higher or lower value of the metabolite may be associated with an increased risk of having or developing pulmonary exacerbations. For specific metabolites, the heatmap provided in FIG. 1C shows whether a higher or lower value of the metabolite is associated with an increased risk of having or developing pulmonary exacerbations.

### Evaluating the Response to Treatment

Another aspect of the present invention provides a method of evaluating the response of a patient with cystic fibrosis having pulmonary exacerbations to treatment. The method includes the steps of determining the level of one or more metabolites associated with pulmonary exacerbations in a sweat sample from the patient; comparing the level of one or more metabolites associated with pulmonary exacerbations to a control value, and characterizing the patient as responding well to treatment if the level of one or more metabolites associated with pulmonary exacerbations has a value closer to the control value.

This method can include embodiments of the invention that correspond to those described for the method of determining the risk of having or developing pulmonary exacerbations. For example, in some embodiments, the method further comprises the step of obtaining a sweat sample from the patient. In further embodiments, the sweat sample is obtained using a skin patch.

In some embodiments, the one or more metabolites are determined using high performance liquid chromatography. In further embodiments, the metabolites associated with pulmonary exacerbations comprise thymidine, N-acetyl tyrosine, 2-piperidinone, and adipate C6-DC. In yet further embodiments, the one or more metabolites associated with pulmonary exacerbations comprise thymidine.

This present aspect of the invention relates to monitoring the treatment of pulmonary exacerbations. A variety of methods are known to those skilled in the art for treating pulmonary exacerbations, and in particular pulmonary exacerbations in patients having cystic fibrosis. Examples of methods of treatment include antibiotic treatment, antiviral treatment, anti-inflammatory (e.g., corticosteroid) treatment, treatment with expectorants, and mechanical ventilation. The use of cysteamine as an adjunct treatment for pulmonary exacerbations of cystic fibrosis has also been described. Devereux et al., PLOS One, 15(12):e0242945 (2020).

In some embodiments, the treatment of pulmonary exacerbations is antibiotic treatment. For a review of antibiotic treatment of pulmonary exacerbations, see Hassani et al., Clin Pharmacokinet., 60(4):447-470 (2021). Examples of suitable antibiotics include β-lactam antibiotics and aminoglycoside antibiotics (e.g., amikacin), with telavancin being a preferred antibiotic for use in treatment of pulmonary exacerbations in cystic fibrosis patients. Additional antibiotics approved for use in treatment of pulmonary exacerbations in cystic fibrosis patients include tobramycin, colistimethate sodium, aztreonam, and levofloxacin.

The method includes the steps of comparing the level of one or more metabolites associated with pulmonary exacerbations to a control value, and the step of characterizing the patient as responding well to treatment if the level of one or more metabolites associated with pulmonary exacerbations has a value closer to the control value. A closer numeric value is a level that is more similar to the control value than the level prior to treatment (e.g., if the control value is 0.5, and the initial value is 1.0, a value of 0.7 is closer, while a value of 1.3 would not be closer). Because the goal is to determine if the patient is responding well to treatment, the goal is for the levels of the metabolites to become closer to the metabolite levels seen in a healthy subject, or a subject who has cystic fibrosis but does not have pulmonary exacerbations. This indicates that the treatment is having the desired outcome. If pulmonary exacerbations cause an increase in the level of the metabolite, a decrease in the level of the metabolite would indicate improvement, while if pulmonary exacerbations cause a decrease in the level of the metabolite, an increase in the level of the metabolite would indicate improvement. In this case, the control value can be the value of the metabolite in a healthy subject, or a subject who has cystic fibrosis but does not have pulmonary exacerbations. For example, the control values can be obtained from the same subject, prior to the development of pulmonary exacerbations.

While in the embodiment described above the metabolite levels are compared with the metabolite levels of a relatively healthy subject, in other embodiments the metabolite levels can be compared with the metabolite levels of the subject before beginning treatment of the pulmonary exacerbations. In such other embodiments, the patient can be characterized as responding well to treatment if the metabolite levels diverge from those seen before treatment. In such a case, the control value would be the levels present before beginning treatment. Again, if pulmonary exacerbations cause an increase in the level of the metabolite, a decrease in the level of the metabolite would indicate improvement, while if pulmonary exacerbations cause a decrease in the level of the metabolite, an increase in the level of the metabolite would indicate improvement.

The following example is included for purposes of illustration and is not intended to limit the scope of the invention.

### EXAMPLE

### Sweat Metabolomics Profiling of Cystic Fibrosis Pulmonary Exacerbations

The inventors sought to characterize the sweat metabolome in PWCF at the time of an acute PEx and again following IV antibiotic treatment to 1) help define biological processes occurring during PEx and 2) develop non-invasive biomarkers predictive of clinical response.

### Methods

### Study Design

We implemented a longitudinal study design collecting a sweat sample at the time of hospitalization for PEx and then again following pharmacological therapeutic intervention (approximately 14 days later). PEx onset was determined by the treating physician, which is guided by published definitions. Rosenfeld et al. J Pediatr., 139:359-65 (2001). PEx resolution occurred between 8 and 21 days (median 13.5 days). PEx resolution was also determined by the treating clinician based on symptom resolution and improvement in lung function measured as FEV1. Baseline FEV1 was determined by their best value in the year prior to PEx not associated with treatment of a recent PEx. Samples were shipped on dry ice to Metabolon, Inc. (Morrisville, NC) where global metabolomics profiles were analyzed using ultra-high-performance liquid chromatography/tandem accurate mass spectrometry (UHPLC/MS-MS). Pre- and post-treatment samples were compared and individual metabolites whose levels differed significantly between samples were identified.

### Subjects

The study was approved by the local Institutional Review Board (IRB14-00890). Potential study participants (aged ≥6 years) were approached in our pulmonary clinic if they had a diagnosis of CF, were experiencing a PEx, and were deemed to be capable of having sweat samples collected. No restrictions of last PEx or minimum FEV1 were employed. Participant clinical information was recorded in a secure electronic database. All participants were taking standard CF medications including pancreatic enzymes, inhaled mucolytics, and fat-soluble multivitamins.

### Sample Collection

Sweat samples were collected using the Wecor Macroduct® Sweat Collection System (ELITech Group, Puteaux, France) according to manufacturer's recommendations. Hammond et al., J Pediatr., 124:255-60 (1994). ˜200 μl of pooled sweat from the left and right forearms was expelled into sterile Eppendorf tubes and immediately flash frozen and stored at -80° C. until processing. The pre-PEx treatment sample collection was obtained on the day of admission prior to initiation of antibiotics, while the post-PEx treatment sample was acquired after completion of antibiotics on the day of discharge. Participants were not fasting at the time of sample collection.

### Metabolomics Processing

Sample processing for metabolomics was performed through Metabolon as previously described (Pediatr Pulmonol., 53(5):583-591 (2018)); Kopp et al., J Cyst. Fibros. 18: 507-515 (2019).

Statistical analysIs

Metabolomics statistical analyses were conducted using log-transformed expression values via MetaboAnalyst (4.0) with the R program and per our prior methods. Kopp et al., J Cyst Fibros., 18:507-15 (2019). A paired t-test was used to identify biochemicals that differed significantly between pre- and post-treatment periods with q values calculated. Within MetaboAnalyst (4.0), partial least squares discriminant analysis (PLS-DA) was performed with 10-fold cross validation. Variable importance on projection (VIP) values of metabolites were obtained to explain the importance of differentially expressed metabolites. Random forest analysis was performed as described to generate biochemical importance plots with class error predictive rates shown. Breiman L. Random Forests. Machine Learning, 45:5-32 (2001). Hierarchical clustering of metabolite profiles was determined using the average clustering algorithm and a Ward's distance measure. Canonical pathway analysis and upstream regulator identification was performed using Ingenuity Pathway Analysis software (IPA) and MetaboAnalyst (4.0). Wisniewski et al., J Cyst Fibros., 19(5), 791-800 (2020). Pathway topology analysis was performed with a node importance measure of relative betweenness centrality with mapping against -log(p) and a false discovery rate <0.05. Metabolite Set Enrichment Analysis (MESA) was performed using the metabolic pathway-associated metabolite sets (99 metabolite sets based on normal human metabolic pathways). Pathway enrichment analysis used the statistical package globaltest 3.12 for human pathways. Goeman et al., Bioinformatics, 20:93-9 (2004). Multivariate exploratory receiver operating characteristic (ROC) curves were generated with linear support vector machine (SVM) analysis utilizing Monte-Carlo cross validation (MCCV) with balanced sub-sampling. ROC performance was measured via a confusion matrix using MetaboAnalyst (4.0) software. FEV1 analysis was performed with paired t-tests between individual time points using GraphPad Prism 8.2. For all analyses two-sided p<0.05 was considered significant.

### Results

### Demographics

Twenty-nine people were enrolled in the study, but only 26 completed both pre- and post-exacerbation samples. Two subjects left the hospital prior to providing a second sweat sample and the other failed to provide sufficient sweat on the first collection due to what we suspect was severe dehydration. Baseline data from these three subjects were excluded from our analyses. The demographics of the 26 people who completed the study are presented in Table 1. The cohort had a higher percentage of females enrolled.

TABLE 1
Baseline Characteristics of the Analysis Cohort*

| | |
|---|---|
| Completed study, n | 26 |
| Gender, female (%) | 73.1 |
| Age (years) | 27.6 ± 10.8 |
| F508del homozygous (%) | 61.5 |
| heterozygous (%) | 34.7 |
| Pseudomonas aeruginosa (%) | 38.5 |
| MRSA (%) | 53.9 |
| % predicted FEV₁ | 64.9 ± 23.4 |
| Body mass index | 21.0 ± 4.8 |
| ^{†}Pancreatic insufficiency (%) | 100 |
| ^{‡}CF-related diabetes (%) | 46.2 |
| Medications | |
| CFTR modulator (%) | 19.2 |
| Psychiatric (%) | 69.2 |
| Proton pump inhibitor (%) | 76.9 |

*Descriptive statistics calculated for subjects contributing pre- and post-PEx treatment samples. Estimates presented as mean ± standard deviation for continuous variables.
^{†}Pancreatic insufficiency defined as treatment with pancreatic enzymes at time of enrollment.
^{‡}CF-related diabetes defined as insulin therapy at time of enrollment.

### Sweat Metabolomics Differentiates PEx States

A total of 326 compounds of known identity were detected in sweat samples. Log-transformed metabolomics profiles were generated to compare profiles before and after PEx. Overall, 147 metabolites were differentially expressed pre-compared to post-PEx. Distinct profiles were seen pre- and post-PEx by supervised clustering (FIG. 1A). The VIP scores are shown for the top 15 metabolites selected as variables for component 1 (FIG. 1B). Ten-fold cross validation of the PLS-DA model demonstrated an overall group classification accuracy of 0.84, coefficient of determination R2=0.85, and cross-validated coefficient of determination Q2=0.46. We then used hierarchical clustering to generate a heat map of the top 25 differentially expressed metabolites between the pre- and post-PEx treatment conditions (FIG. 1C). A Euclidean clustering algorithm with Ward's method separated most samples based on these top 25 metabolites. The pre-PEx state was associated with increased expression of metabolites such as thymidine and leucylglycine and decreased expression of threonate and 2-piperidone while post-PEx samples demonstrated reverse trends for these same metabolites (FIG. 1C).

We internally validated these findings with a random forest analysis to generate a biochemical importance plot. Top metabolites are displayed by increasing importance for separation of the two experimental conditions (FIG. 2A). Metabolites are color-coded by general groups such as amino acids, lipids, nucleotides, etc. The top metabolites involved amino acid metabolism, lipid metabolism, and peptides. Like our prior heatmap, thymidine was the top metabolite identified for separating pre- and post-PEx states. The random forest model yielded a predictive accuracy of 79% (FIG. 2A).

### Pathway Analysis of Sweat Metabolomics Profiles

Next, we defined biologically relevant patterns in the differentially expressed metabolites. We utilized the MetaboAnalyst pathway analysis module with pathway topology mapping to define relevant patterns post-PEx compared to pre-PEx (FIG. 2B). Pathways are shown as nodes with varying significance based upon color and size. Selected pathway nodes of importance are annotated. Post-PEx was associated with changes in pyrimidine, phenylalanine, glycerolipids, glycine, taurine, arginine, and alanine metabolism (FIG. 2B).

We then used IPA to further analyze pathways impacted by PEx state. L-amino acid oxidase 1 (Lao1) and IL-37 were identified as the top upstream regulators of predicted altered metabolic pathways during PEx (both predicted to be inhibited, FIG. 3). Lao1 is involved in amino acid catabolismlL-37 regulates both innate and adaptive immune responses. Activated metabolites downstream of Lao1 and IL-37 were predicted to lead to activation of pathways necessary for bacterial growth and albumin synthesis, while inhibiting uptake of L-alanine and D-glucose (FIG. 3). Top canonical pathways predicted to be altered between pre- and post-PEx are shown in Table 2. These included tRNA charging, purine degradation, adenosine degradation, and the urea cycle.

TABLE 2
Top Canonical Pathways altered between pre- and post-PEx

| Pathway name | p-value | Overlap (%) |
|---|---|---|
| tRNA Charging | 2.39E−11 | 44.2% 19/43 |
| Purine Nucleotides Degradation II (Aerobic) | 5.49E−08 | 58.8% 10/17 |
| Adenosine Nucleotides Degradation II | 1.20E−07 | 72.7% 8/11 |
| Purine Ribonucleosides Degradation | 3.38E−07 | 66.7% 8/12 |
| to Ribose-1-phosphate | | |
| Urea Cycle | 2.69E−05 | 50.0% 7/14 |

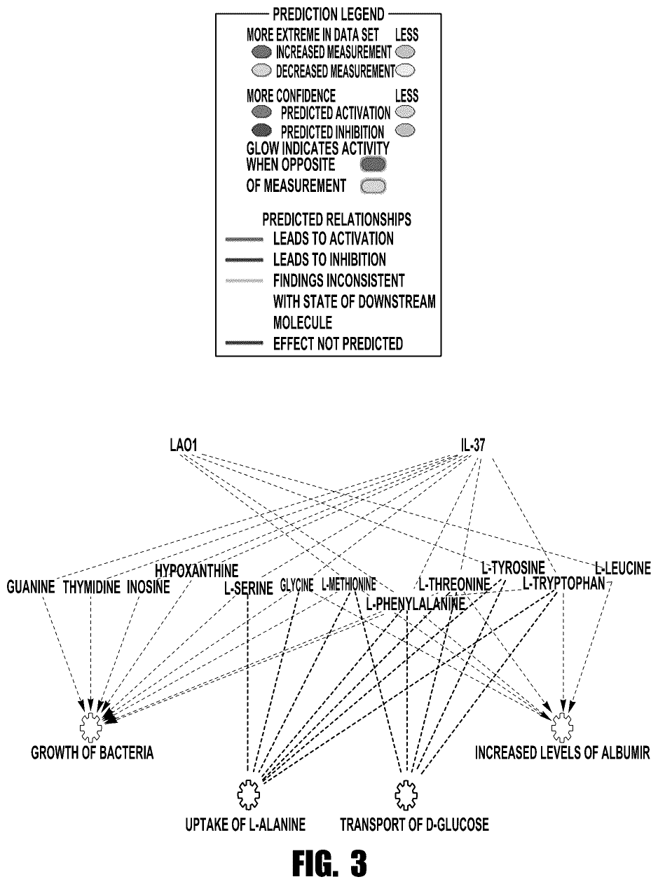

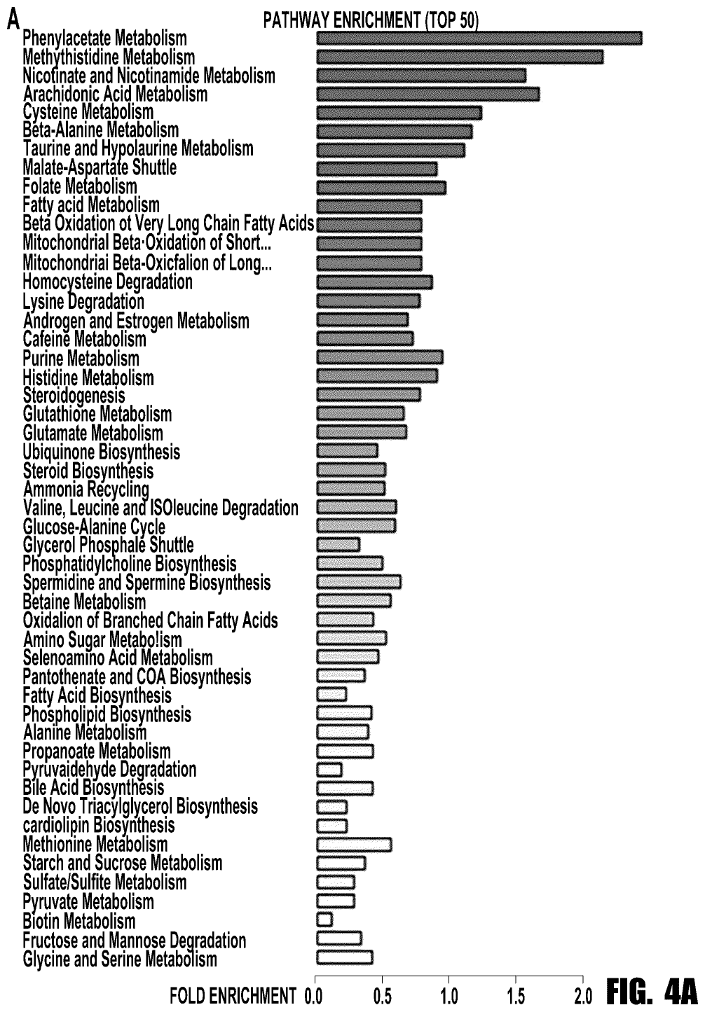

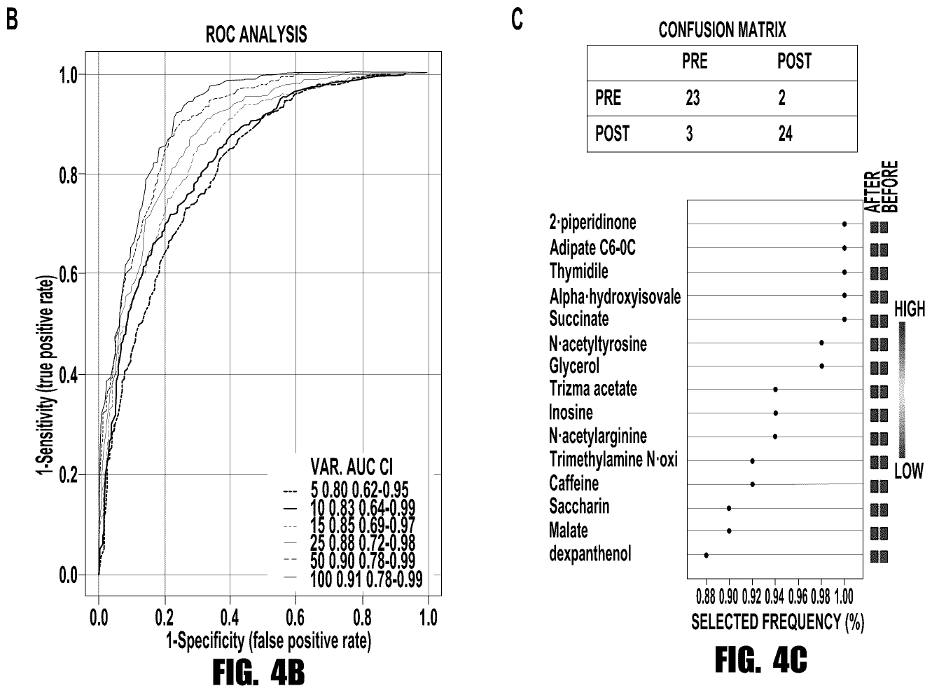

To further interrogate altered pathways, we used MESA (99 metabolite libraries) to distinguish enriched pathways between pre- and post-PEx. MESA analysis showed multiple significantly enriched pathways that occurred between PEx states (FIG. 4A). Top significantly enriched pathways included phenylacetate metabolism, methylhistidine metabolism, nicotinate metabolism, arachidonic acid metabolism, and cysteine metabolism. Combined, multiple pathway analyses demonstrated a wide-ranging effect of PEx on metabolic states in CF.

### Sweat Metabolomics Biomarkers

Due to the unique differences in metabolite expression between pre- and post-PEx states, we used a multivariate ROC analysis using linear SVM to identify potential metabolite biomarkers to distinguish between clinical states. ROC curves were generated by 50 iterations of MCCV using balanced sub-sampling to estimate the predictive performance and the stability of the selected features. The first 2/3 of the samples were used to evaluate feature importance and select important features to build models and the remaining 1/3 of the samples were used to validate these models. The predictive performance of these models was assessed by area under the ROC curve (AUC). ROC curves for 5-100 metabolites based on cross-validation performance are shown in FIG. 4B, with the average of all model curves displayed. The ROC curves showed strong predictive capabilities, with AUC ranging from 0.802 (5 variables, CI 0.62-0.95) to 0.913 (100 variables, CI 0.78-0.99). A confusion matrix performance evaluation accurately distinguished pre- and post-PEx based on the models (FIG. 4C). The top 15 metabolites utilized in the ROC analysis are displayed (FIG. 4C).

### Metabolomics Analysis by Failure to Return to Baseline FEV1

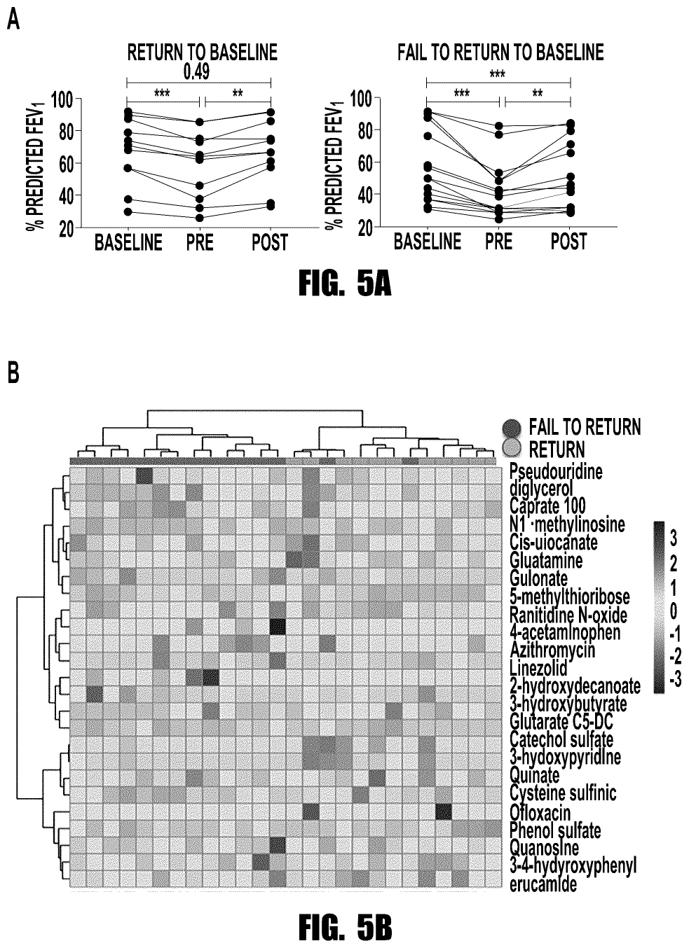

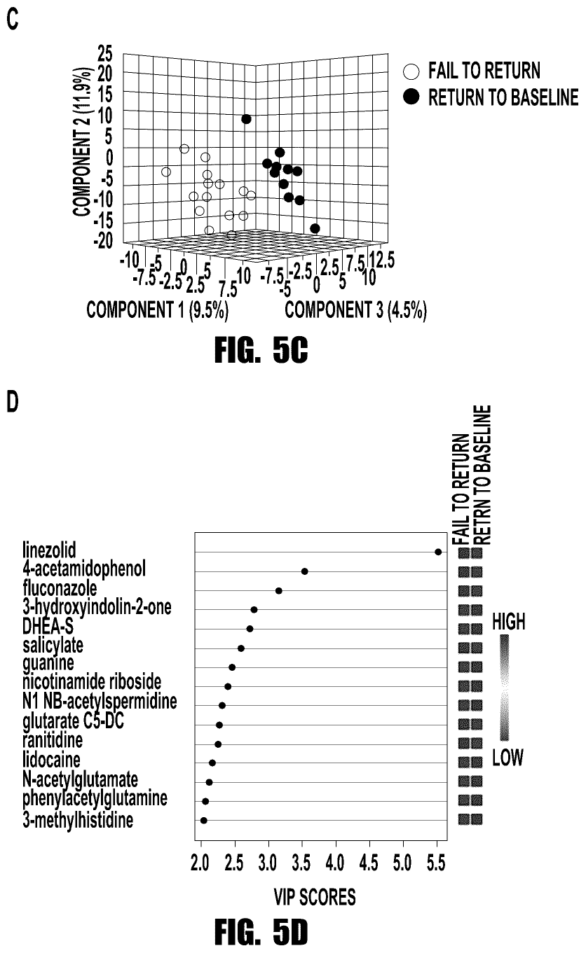

Within our cohort, 15 people failed to return to within 5% of their baseline % predicted FEV1 following intravenous antibiotic treatment (FIG. 5A). Therefore, we conducted a separate analysis to determine if sweat metabolomes could distinguish between those that returned to their baseline FEV1 and those that did not. We used hierarchical clustering to generate a heat map of the top 25 differentially expressed metabolites between people who returned to baseline (return) and those that failed to return to baseline FEV1 (fail to return) (FIG. 5B). All but 2 fail to return samples grouped together using this method. Fail to return to baseline was associated with increased expression of pseudouridine and glutamine (FIG. 5B). We then used PLS-DA to further classify differences in group metabolite profiles and determine VIP scores (FIGS. 5C-D). While this PLS-DA model had a quite high R2 of 0.99, it had low accuracy and Q2, which were 0.58 and 0.09, respectively. These results suggest over-fitting of this model.

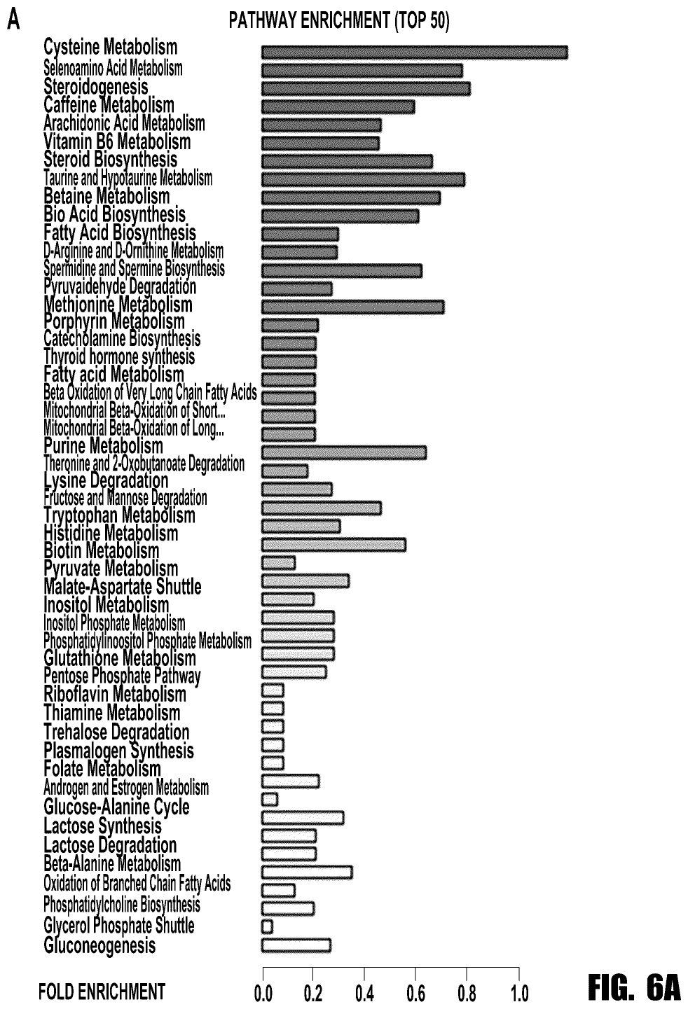

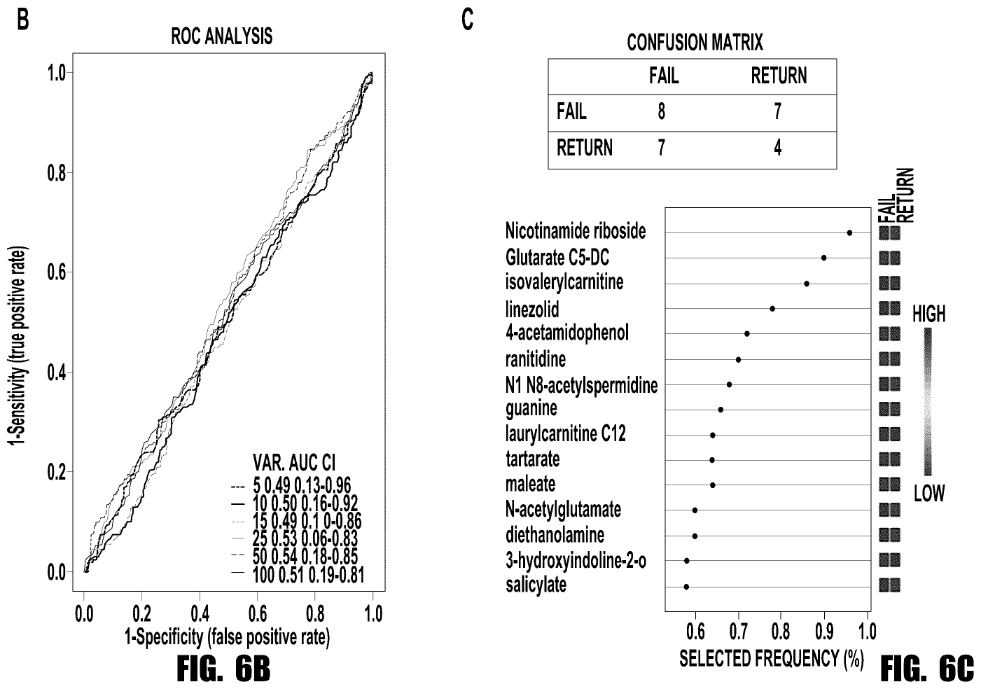

We then conducted MESA pathway analysis between return and fail to return to baseline FEV1 groups. However, only cysteine metabolism was significantly enriched in those who failed to return to baseline FEV1 compared to those who returned to baseline (FIG. 6A). Lastly, we performed ROC analysis to determine potential biomarkers to distinguish these groups. ROC curve models lacked predictive abilities, with AUCs ranging from 0.486 (5 variables, CI 0.13-0.96) to 0.541 (50 variables, CI 0.18-0.85) (FIG. 6B). A confusion matrix performance evaluation confirmed a lack of predictive ability (FIG. 6C). The top 15 metabolites utilized in the ROC analysis are displayed (FIG. 6C).

### Discussion

Host, environmental, social factors, and adherence to long-term therapies influence the relationship and progression of infection and inflammation that can precipitate PEx episodes in PWCF. PEx occur frequently in PWCF and are associated with a loss of FEV1, reduction in quality of life, and an increase in mortality. McLeod et al., J Cyst Fibros. 19(6), 858-867 (2020). Previous studies have identified gene expression patterns of different host cells associated with PEx, (Stachowiak et al., J Clin Med., 16;9(6):1887 (2020)) but the metabolomic pathways affected by PEx are not well defined. Using sweat collected at two times points, the present study completed a comprehensive assessment of metabolomic differences at the onset of a PEx in PWCF and then after completion of an antibiotic treatment course. Our findings show sweat metabolomes were distinctly different between PWCF at the time of a PEx and after completion of the therapeutic intervention. Additionally, distinct sweat metabolomes were discovered in PWCF who failed to return to their baseline pulmonary function post-PEx treatment. Expanding our understanding of metabolites and related pathways along with alterations in CF during a PEx may be useful for future efforts in long-term management. Such efforts could include real-time health monitoring, identification of intervention points at the time of a developing PEx and identifying those at risk of further pulmonary decline.

In contrast to our approach, other investigators have performed metabolomic profiling in PWCF with blood, which is accessible in a clinical setting. Al-Qahtani et al., J Proteome Res., 19:2346-57 (2020). Biomarkers of CF lung disease are influenced by the challenges of obtaining relevant and timely clinical samples. Ramsey et al., Paediatr Respir Rev., 16:213-8 (2015). To really take advantage of metabolomic profiling technology as it evolves, we believe clinical sampling in PWCF needs to be collected non-invasively, so patients can be monitored in real-time, potentially even at home or other social settings such as school or workplace. Acquisition of blood for metabolomic profiling as a means of surveillance of health is invasive with associated pain and challenging to do outside of a clinical setting. Although sweat has limited clinical use currently, recent technological advancements make it an appealing biofluid for future development for clinical indications. Mena-Bravo A, Luque de Castro M D., J Pharm Biomed Anal., 90:139-47 (2014) The first characterization of the sweat metabolome in PWCF was reported in infants that compared confirmed CF diagnoses with carriers of a CFTR mutation. As with the current study, these investigators used a currently available clinical device to generate and collect sweat with a non-targeted metabolite profiling approach that identified metabolite differences in the two infant cohorts. Macedo et al., ACS Cent Sci., 3:904-13 (2017). In another study using an age- and gender-matched healthy control group, PWCF ranging from 5 to 19 years old were found to have sweat metabolomic differences in targeted molecular classes (bile acids, a glutaric acid derivative, thyrotropin-releasing hormone, an inflammatory mediator, a phosphatidic acid, and diacylglycerol isomers) using skin imprints on silica plates used to collect sweat. Esteves et al., Front Pediatr., 5:290 (2017). To our knowledge, our study if the first characterization of sweat metabolomics in PWCF to characterize metabolites and their profiles at two different times points prior to initiating antibiotics for a PEx and upon completion of antibiotics.

An important aim of this study was to expose and define the biological processes that were altered during PEx. By comparing the metabolites that were present at the time of PEx and then again following completion of antibiotic treatment (approximately 14 days later), we unveiled several metabolic pathways that may be affected by increased microbial pulmonary burden and resultant inflammation. It is important to note that PEx treatment consistently includes antibiotics that target organisms detected in current and previous culture testing, chest physiotherapy, aerosol therapy, nutritional support with high protein and high calorie diets, and daily physical therapy. As a result of treatment, sweat analyses showed marked fluctuations [upregulated or down regulated] in specific metabolic intermediates within pathways that included phenylacetate metabolism (urea cycle defects), methylhistidine metabolism (product of peptide bond synthesis and methylation of actin and myosin), nicotinate and nicotinamide metabolism (important for ramping up NAD and NADPH biosynthesis for energy metabolism), arachidonic acid metabolism (production of prostaglandins and leukotrienes—proinflammatory), cysteine metabolism (production of essential metabolites, antioxidation), and taurine and hypotaurine metabolism (conjugation of bile acids, antioxidation, osmoregulation, membrane stabilization, and modulation of calcium signaling).

We have previously reported enrichment of pathways involving cysteine metabolism and its metabolites (taurine and hypotaurine) in infants and children with CF and exposure to secondhand smoke. Wisniewski B L, Shrestha C L, Zhang S, Thompson R, Gross M, Groner J A, et al., J Cyst Fibros. 19(5), 791-800 (2020). However, objective measures of tobacco exposure were not available for this study. Several studies have shown that cysteamine (a metabolite precursor to hypotuarine) decreases proinflammatory molecules, inhibits biofilm formation, and facilitates autophagy and macrophage-assisted destruction of bacteria. Charrier et al., Orphanet journal of rare diseases, 9:189 (2014) The results of a phase II trial of cysteamine to test its efficacy during PEx showed a reduction in symptoms, significantly reduced chronic respiratory infection symptom score and reduced white blood cell count, and reduced CRP. Devereux et al., PLOS One, 15:e0242945 (2020) In the subset of PWCF who failed to return to baseline lung function after PEx treatment, cysteine metabolism was also significantly enriched. This suggests that although pathways of dysregulated metabolism may be shared amongst PWCF experiencing PEx, specific metabolic derangements may be common to those who did not return to a clinical baseline following hospitalization. Further studies will need to examine such relationships in larger, longitudinal cohorts as well as determine other associations that were unable to be determined by our limited sample size for the return to baseline analysis.

Interestingly, our results also showed enrichment of pathways involving arachidonic metabolism following treatment. Dysregulation of arachidonic acid metabolism during PEX and poor growth has been previously reported, (Freedman et al., The New England journal of medicine, 350:560-9 (2004)) as well as the overall contribution of altered arachidonic acid metabolism to inflammation in CF. We also recently demonstrated that dysregulated arachidonic acid metabolism in children with CF is associated with impaired microbial clearance. Kopp et al., Thorax, 74(3):237-246 (2019). We hypothesize that the pathophysiology of PEx results in further disruption of abnormal arachidonic acid metabolism, which contributes to heightened inflammation and impaired microbial clearance during PEx. Metabolomics monitoring of specific metabolic pathways is a potential approach for identifying impending PEx and monitoring the impact of new therapeutics.

The current study has several limitations. A single-center study can induce bias. Sample collection for our experiments did not include a collection point for metabolomics analysis prior to the PEx, which would have established metabolite profiles prior to the PEx. Therefore, we do not have a baseline metabolomics profile of our cohort prior to PEx to determine causality. Due to inherent constraints of our hospital, we could not recruit an age-matched healthy control group for comparison, which can limit our analyses. There are also inherent constraints in the analytics employed due to the relatively small sample size, which warrant future studies. However, complementary analyses employed in the current study internally validate our findings. A limited number of PWCF in our study were on CFTR-modulator therapy at the time of sample collection, so the effect of CFTR modulators on metabolite pathways important in PEx was not determined. Similarly, dietary and other pharmacologic factors can influence metabolomics analysis in CF. All PWCF were on high protein-high calorie diets in accordance with CF Foundation guidelines and used similar systemic therapies such as pancreatic enzymes and fat-soluble vitamins per prior studies. Wisniewski et al., J Cyst Fibros., 19(5):791-800 (2020). A unique limitation of sweat metabolomics studies would be confounding metabolite analysis through skin care product use. A hospital-based design allowed us to control a limited number of skin care products (e.g. moisturizers) used in this study. Overall, the potential limitations of the study are balanced by the new information gained.

In conclusion, PEx alters sweat metabolomics in PWCF and may serve as a useful diagnostic tool to improve identification and treatment of PEx. Our analysis found important pathway alterations occurring in PEx, which need future studies to enhance our understanding of the pathobiology of PEx in PWCF. Moreover, a non-invasive means such as sweat collection to diagnose PEx, especially early in the process of its development, would be very beneficial for both clinical and research purposes.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of determining if a patient having cystic fibrosis has an increased risk of having or developing pulmonary exacerbations, comprising:
determining the level of one or more metabolites associated with pulmonary exacerbations in a sweat sample from the patient, and
characterizing the patient as having an increased risk of having or developing pulmonary exacerbations if the level of one or more metabolites associated with pulmonary exacerbation are significantly different from a control value.

2. The method of claim 1, further comprising the step of obtaining a sweat sample from the patient.

3. The method of claim 2, wherein the sweat sample is obtained using a skin patch.

4. The method of claim 1, wherein the level of the one or more metabolites is determined using high performance liquid chromatography.

5. The method of claim 1, wherein the metabolites associated with pulmonary exacerbations are selected from the group consisting of thymidine, N-acetyl tyrosine, 2-piperidinone, and adipate C6-DC.

6. The method of claim 1, wherein the one or more metabolites associated with pulmonary exacerbations comprises thymidine.

7. A method of evaluating the response of a patient with cystic fibrosis having pulmonary exacerbations to treatment, comprising:
determining the level of one or more metabolites associated with pulmonary exacerbations in a sweat sample from the patient;
comparing the level of one or more metabolites associated with pulmonary exacerbations to a control value, and
characterizing the patient as responding well to treatment if the level of one or more metabolites associated with pulmonary exacerbations has a value closer to the control value.

8. The method of claim 7, further comprising the step of obtaining a sweat sample from the patient.

9. The method of claim 8, wherein the sweat sample is obtained using a skin patch.

10. The method of claim 7, wherein the one or more metabolites are determined using high performance liquid chromatography.

11. The method of claim 7, wherein the metabolites associated with pulmonary exacerbations comprise thymidine, N-acetyl tyrosine, 2-piperidinone, and adipate C6-DC.

12. The method of claim 7, wherein the one or more metabolites associated with pulmonary exacerbations comprise thymidine.

13. The method of claim 6, wherein the treatment is antibiotic treatment.
